(19) United States
(12) Patent Application Publication
(10) Pub. No.: US 2021/0005325 A1
(43) Pub. Date: Jan. 7, 2021

(54) INTEGRATED SEARCHING OF NON-MEDIA DATA AND MEDIA DATA IN CAMPAIGN PLANNING

(71) Applicant: DEEPINTENT, INC., New York, NY (US)

(72) Inventors: JENNIFER WERTHER PERLMAN, HILLSDALE, NJ (US); CHRISTOPHER T. PAQUETTE, NEW YORK, NY (US)

(21) Appl. No.: 16/503,256

(22) Filed: Jul. 3, 2019

(51) Int. Cl. G16H 50/70; G16H 70/40; G16H 70/20; G16H 10/60; G06F 16/2458; G06F 16/23; G06F 16/29; G06F 16/2457

(52) U.S. Cl. CPC G16H 50/70; G16H 70/40; G16H 70/20; G06F 16/24578; G06F 16/2465; G06F 16/23; G06F 16/29; G16H 10/60

(57) ABSTRACT

A computer implemented method for selecting targeted digital impressions based upon unifying digital identity data for computers, devices, accounts or users to data source from third parties that can support defining targeted audiences for campaigns and delivering highly relevant impressions to members of the target audiences. A DSP campaign planning system receives one or more NPIs of one or more HCPs, practice data indexed by the NPIs, and one or more of prescription data specifying drug prescriptions previously written and indexed by the NPIs and/or clinical medical data indexed by the NPIs. The practice data, prescription data, clinical medical data, medical claims data, demographic data, practice/specialty data, address data, education data or other datasets may be mapped to or associated with NPI values in a database of digital identity data such as cookies or device IDs shared by the DSP campaign planning system and a DSP. The DSP campaign planning system supports defining target audiences, based upon the NPIs, practice data associated with the NPIs, and one or more of the prescription data, the clinical medical data, and the medical claims data, representing a campaign plan for targeting digital impressions to a specified HCP audience segment. Such campaigns may be executed based upon matching NPIs of user devices to cookie data represented in the plans. Audience data is transmitted to a DSP programmed to transmit digital instructions to an advertising exchange server for executing one or more targeted digital impressions via a media and advertisement display channel and user computer. Reporting and measurement functions may be programmed as part of the DSP campaign planning system.

### TECHNICAL FIELD

One technical field of the disclosure is computer implemented demand-side platform (DSP) systems, which are used in digital advertising technology. Another technical field is relational databases and specifically the use under stored program control of automatic joins of tables that store different datasets.

### BACKGROUND OF DISCLOSURE

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

Digital advertising technology (ad tech) uses distributed computer systems under stored program control to determine what media or contents that user computers are accessing, as well as what digital advertising units to select and transmit or place in media, content or other locations. Ad tech systems have developed sophisticated means for bidding on the placement of electronic ad units within websites, mobile device feeds and other applications. However, present ad tech systems still suffer from many limitations.

Many advertising agencies, pharmaceutical companies, medical equipment companies, insurance companies and other healthcare related firms wish to enhance advertising impressions of healthcare products and services to the applicable healthcare providers (HCPs). Impression deployment can entail demand side platform (DSP) systems for targeted distribution of product information. Determining the appropriate online HCP identities and where to deliver information regarding specific products and services can be challenging given the myriad types of medical conditions, HCPs, their practice histories, and the multitude of different products in the healthcare industry. Clinical medical data, prescribing behavior data, National Provider Identifier (NPI) data, demographic, certification, appointment scheduling, payment data and other information relating to a HCP's NPI is not generally accessible to agencies for use in determining which HCPs would be best fit for distributing information pertaining to particular products or may be outdated, not fully comprehensive and not coordinated with other data, and therefore limited in its utility. Thus, DSP systems often distribute product information to HCPs whose patients would not benefit from such distribution and/or omit distribution to many HCPs whose patients would benefit.

Data sellers are known to sell data defining audience segments into a DSP like The Trade Desk. These approaches usually allow for only minimal customization of the audience to be targeted and rely on buckets or segments of cookie or device data that have been manually tagged to indicate a particular audience characteristic. Other data providers offer data via platforms which provide counts and aggregations for how many users with various attributes are recorded in a database of HCPs; these platforms do not have a DSP and require an intermediary to transfer audience data to a DSP. The lack of integration in this approach precludes providing HCP-specific reporting of engagement with advertisements in real-time. Furthermore, existing systems may use individual data stores based on browser cookie limitations and provide no sound way to unify digital identity data with third-party data.

This disclosure addresses the technical problem of how to automatically join and/or correlate disparate datasets of healthcare data in conjunction with digital presence data relating to HCPs to find better ways of transmitting relevant content to these parties in real time, including providing distribution costs and performance data. There is also a need for better tools for planning campaigns in terms of creating clinically relevant audiences, cost and for obtaining post-campaign performance data.

### BRIEF DESCRIPTION OF THE DRAWINGS

In the drawings:

FIG. 1 illustrates a demand side platform (DSP) process directed to health care providers (HCPs) according to embodiments.

FIG. 2 illustrates a DSP process according to embodiments.

FIG. 3 illustrates a DSP campaign plan, configuration and implementation process according to embodiments.

FIG. 4A illustrates a computer-generated graphical user interface (GUI) of a DSP campaign planning tool according to embodiments.

FIG. 4B illustrates a computer-generated GUI that is programmed for selecting DSP campaign filter parameters to define clinically relevant audiences using diagnosis and procedure data according to embodiments.

FIG. 5A illustrates a computer-generated GUI display that is programmed for reviewing DSP campaign planning data of HCP specialties according to embodiments.

FIG. 5B illustrates a computer-generated GUI display that is programmed for reviewing DSP campaign planning data of HCP geography according to embodiments.

FIG. 5C is a GUI display for reviewing target HCP data by decile grouping according to embodiments.

FIG. 5D is a GUI display for reviewing HCP clinical/diagnosis code data for DSP planning according to embodiments.

FIG. 5E is a GUI display for reviewing HCP clinical/procedure code using CPT or HCPCS or other codes for DSP planning according to embodiments.

FIG. 5F is a GUI display for showing targeted HCP data based upon one or more custom fields.

FIG. 6 is a block diagram that illustrates an example computer system with which embodiments may be implemented.

### DETAILED DESCRIPTION

Embodiments include methods and systems for selecting and processing digital impressions for healthcare providers (HCPs). In embodiments, a demand side platform (DSP) is implemented utilizing computer servers that are configured to receive datasets including but not limited to: National Provider Identifiers (NPIs) of HCPs; primary and non-primary specialty or practice area; demographics such as age, gender and location; best-of-breed (BOB) address; education. “Non-primary,” in this context, may refer to a secondary specialty, tertiary specialty and so forth. These datasets may be sourced from a combination of public databases and third-party datasets. Some embodiments may receive and use other data, such as clinical, medical claims, and/or prescription data indexed by the NPIs. Based upon the received data and configuration of DSP parameters and filters by the agency, digital distribution instructions are generated by the DSP. In various embodiments, the instructions include distributing digital advertisements to an advertising exchange server for implementing targeted digital impressions to HCPs.

In one embodiment, the disclosure provides a computer implemented method for transmitting targeted digital impressions to a filtered audience, the method comprising receiving, using one or more computers programmed to implement a demand side platform (DSP) campaign planning system, a first list of one or more healthcare provider identifiers each uniquely identifying healthcare providers; receiving, at the one or more computers, practice data comprising one or more of: clinical medical data indexed by the healthcare provider identifiers, prescription data specifying drug prescriptions previously written and indexed by the healthcare provider identifiers and/or medical claims data indexed by the healthcare provider identifiers and storing the data in a shared database; displaying, in a graphical user interface that is generated by the DSP campaign planning system and provided to one or more client computers, counts of healthcare providers based upon using the first list and matching healthcare provider identifiers in the first list to the practice data; receiving, from one or more client computers, input specifying one or more filter attributes, and in response, filtering the first list based on the one or more filter attributes to produce a filtered second list of healthcare provider identifiers representing a target audience of healthcare provider identifiers; transmitting the filtered second list of healthcare provider identifiers to a DSP service implemented using one or more DSP server computers, the DSP service being programmed to receive a particular healthcare provider identifier, match the particular healthcare provider identifier to the filtered second list, and in response to a match, generate instructions for use by one or more of an advertising exchange, media server and/or media and advertisement display channel to cause presentation of a targeted digital impression on a user computer associated with the particular healthcare provider identifier.

In one feature, the healthcare provider identifiers comprise any of United States National Provider identifiers (NPIs), CINC values or Practitioner Identifiers. In another feature, the method further comprises receiving, at the one or more computers, for use in association with the practice data, one or more of demographic data, specialty data, best-of-breed address data and/or education data, each being indexed by the healthcare provider identifiers. In another feature, the practice data comprises at least one of diagnosis, procedure, or prescription history associated with the healthcare provider identifiers. In a further feature, the clinical medical data comprises practice and patient statistics related to at least one of HCP specialty, geography, or HCP claim codes. In yet another feature, the method further comprises repeating, two or more times, the steps of displaying in a graphical user interface that is generated by the DSP campaign planning system and receiving input specifying one or more filter attributes, thereby repeatedly updating the filtered second list according to different filter attributes.

In a further feature, the method further comprises generating and displaying a graphical user interface that is programmed to receive client computer input specifying filter attributes for clinical data by diagnosis or procedure, for specialty by primary or non-primary specialty, for geography, for deciles of the healthcare provider identifiers, for diagnosis codes, and for procedure codes. In another feature, the DSP service is programmed for generating instructions for ranking impressions based upon one or more of target HCP practice specialties, target HCP geography, target HCP procedure codes, counts of unique HCPs, counts of unique HCP patients, or estimated numbers of impressions; generating instructions for submitting bids for purchasing impressions based upon the ranking of the impressions. In still another feature, the DSP service is programmed for generating instructions for ranking impressions based upon one or more of target HCP practice specialties, target HCP geography, target HCP procedure codes, counts of unique HCPs, counts of unique HCP patients, or estimated numbers of impressions; generating instructions for submitting bids for purchasing impressions based upon the ranking of the impressions; and the method further comprises receiving, across a computer network, results of impressions that were generated in response to the digital publicizing instructions; based upon the results of impressions, revising instructions for purchasing impressions; transmitting the revised instructions to an advertising exchange server for executing one or more targeted digital impressions.

Example embodiments are described below in sections according to this outline:

1. STRUCTURAL EXAMPLE

2. FUNCTIONAL EXAMPLE

3. IMPLEMENTATION EXAMPLE—HARDWARE OVERVIEW

4. EXTENSIONS AND ALTERNATIVES

### 1. Structural Example

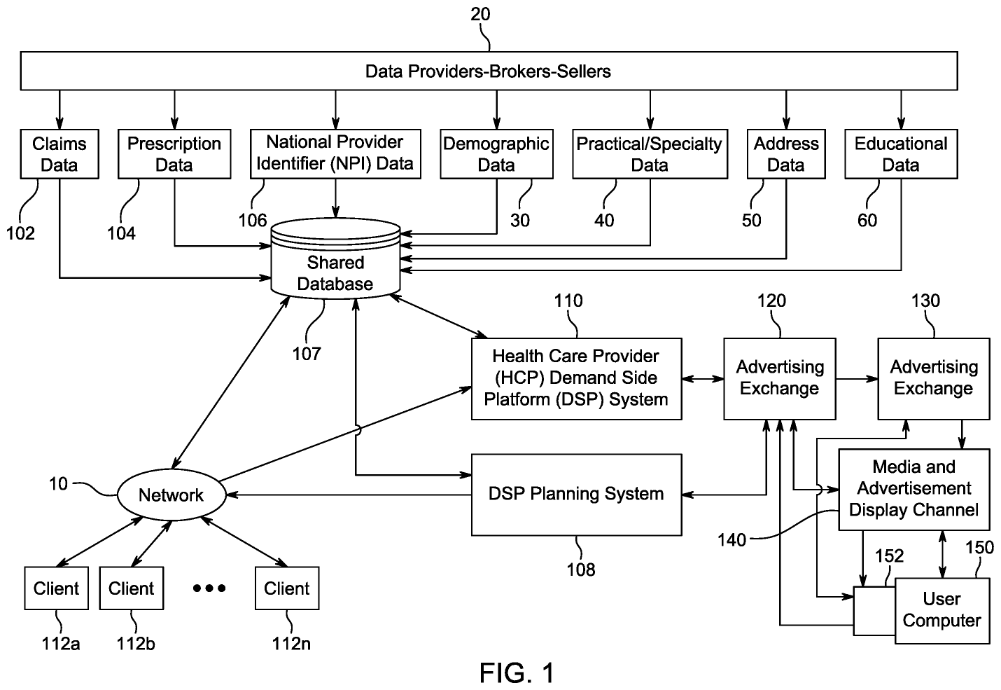

FIG. 1 illustrates a demand side platform (DSP) system directed to health care providers (HCPs) according to embodiments.

Distributed computer systems may be used to implement embodiments. In one embodiment, a plurality of datasets such as claims data 102, prescription data 104, National Provider Identifier (NPI) data 106, demographic data 30, practice/specialty data 40, address data 50, and education data 60 are obtained from data providers-brokers-sellers 20 and persisted to a shared database 107. In some embodiments, one or more of demographic data 30, practice/specialty data 40, address data 50, and education data 60 may be used collectively and termed “demographic data” because the one or more datasets relate to a practice of an HCP.

Shared database 107, a DSP system 110, a DSP campaign planning system 108 and one or more client computers 112a, 112b, 112n are communicatively coupled directly or indirectly through other network or data communication links to a network 10, which broadly represents any one or more of a local area network, wide area network, campus network or internetwork(s) using terrestrial, satellite, wired or wireless links. The shared database 107 thus is directly or indirectly coupled to a DSP system 110 which is operated by one or more server computers or other servers, located on the premises of an enterprise or implemented using virtualized servers, containerization or other facilities of a cloud computing facility. DSP 110 also is communicatively coupled to directly or indirectly to the DSP campaign planning system 108, which executes using the same servers as DSP 110 or different servers and is programmed to implement planning tasks such as defining audience segments, campaigns and scheduling, as further described in other sections herein. DSP campaign planning system 108 also is coupled to any number of client computers represented by client 112a, 112b, 112n in FIG. 1.

DSP 110 is coupled via a network link to an advertising exchange 120, which is coupled to a media server 130. In an embodiment, media server 130 is programmed to media and advertisement display channel 140 that may be direct digital impressions, media or advertisements to digital visual display devices of networked end stations such as user computer 150, which may be a desktop computer, mobile computer, tablet, smartphone or any other device capable of digital electronic visual display. In an embodiment, advertising exchange 120, media server 130, and media and advertisement display channel 140 are communicatively coupled to network 10.

In some embodiments, data from different clients 112a, 112b, 112n is stored in shared database 107 using security controls, access controls or tenant segmentation consistent with HIPAA, client confidentiality policies or other policies. In various embodiments, physically separate or logically segregated data stores may be implemented, for example using multi-tenancy techniques. In this manner, different users of different clients 112a, 112b, 112n at different agencies may have access limited to specified brands, or otherwise access data only subject to access control mechanisms implemented in software.

In an embodiment, DSP 110 is programmed to retrieve or obtain claims data 102, prescription data 104, National Provider Identifier (NPI) data 106, demographic data 30, practice/specialty data 40, address data 50, education data 60 and/or other datasets from data providers-brokers-sellers 20 through shared database 107. Each of claims data 102, prescription data 104, National Provider Identifier (NPI) data 106, demographic data 30, practice/specialty data 40, address data 50, and education data 60 and data providers-brokers-sellers 20 may represent separate data repositories, storage systems, server computers or servers that are accessible programmatically using request-response protocols that are transmitted over networks. In some embodiments, data providers-brokers-sellers 20 may be implemented as switchboards or clinical information exchanges. Datasets can be retrieved, for example, under program control or direction of client 112a, by loading from a spreadsheet or manual data entry, or transmitted across a network such as from a data repository of NPIs. For example, institutional server computers of government agencies or other third parties may expose APIs that DSP system 110 can call to retrieve a response comprising a dataset of NPIs. The particular manner in which NPIs are imported from government or other institutional sources is not critical and what is important is that DSP system 110 acquires digital values corresponding to NPIs 106 by automated or manual means and has a way to store the NPIs for local use. Datasets obtained via data providers-brokers-sellers 20 may be mapped to or associated with NPI values.

In embodiments, medical claims data 102 is obtained from one or more health insurance carriers and prescription data 104 is obtained from one or more pharmacy entities, hospitals, medical groups or from carriers. In other embodiments, both medical claims data 102 and prescription data 104 are obtained from service providers who obtain the data from sources such as carriers, pharmacies, medical groups or others under contract and data transfer agreements with those entities. In an embodiment, the DSP campaign planning system 108 is programmed to obtain data from data providers-brokers-sellers 20 using flat files, spreadsheets or other digital formats for storing raw data from the providers. Additionally or alternatively, DSP campaign planning system 108 is configured with an application programming interface (API) that can interface with such service providers or other data providers across a computer network using programmatic requests to retrieve or download any of the data 102, 104, 106, 30, 40, 50, 60. The specific means of obtaining such data is not critical and what is important is that DSP system 110 acquires digital values corresponding to medical claims data 102, prescription data 104 and any of the other data 106, 30, 40, 50, 60 by automated or manual means and has a way to store the data for local use.

In an embodiment, when received from service providers or data providers, datasets shown in FIG. 1 such as the medical claims data 102 and prescription data 104 includes an NPI to reference the HCP who provided the diagnosis or wrote the prescription represented in the medical claims data 102 and prescription data 104. NPIs may be associated with datasets 30, 40, 50, 60 as well, either by data providers-brokers-sellers 20 or by independent computation at DSP campaign planning system 108. The datasets of FIG. 1 may be stored in the shared database 107 of DSP system 110 using a relational database in which NPI is a primary key and serves to associate the NPIs 106 with values obtained from claims data 102, prescription data 104, demographic data 30, practice/specialty data 40, address data 50, and education data 60. In many cases the datasets received from data sources are associated with NPI values and therefore creating associations in the database 107 can be executed using queries for NPI values in the database and updating records from the datasets.

The DSP system 110 can be accessed by client systems 112a, 112b, 112n which can include marketers of healthcare products and services wishing to use the DSP system to distribute content such as digital advertisements about these products and services to selectively targeted HCPs. Although a limited number of client systems are shown in FIG. 1, in practical embodiments there may be thousands or millions of client systems interacting with the DSP system 110 and the designation “112n” is used to indicate that the number n of such systems is not limited. Each client system 112a, 112b, 112n comprises a computer or process associated with a user account that has been opened in the DSP system 110 and user accounts typically are associated with persons at brands or advertisers.

The client systems 112a, 112b, 112n select and configure parameters within the DSP system in order to plan a distribution strategy and generate instructions for deployment of the strategy through an advertising exchange server system 120. In embodiments, the selected parameters can include diagnosis and procedure codes such as ICD-10 codes for diagnoses; codes for procedures such as Current Procedural Terminology (CPT) codes associated with HCPs, HCPCS codes, J codes; NDC codes for prescriptions; as well as HCP specialties, geographies, classes of medications and/or devices used by HCPs to which the content is directed. In embodiments, the DSP system 110 can receive and analyze impression data from the advertising exchange system 120 about different impressions provided across a distribution network. In some cases, impression data may be associated with values of cookie 152, the use of which is described further in other sections. For purposes of illustrating a clear example, a cookie 152 is shown in FIG. 1 but other embodiments may use, additionally or as an alternative to cookies, a mobile advertising identifier, third-party identity token, or other digital identity value that is capable of use in digital advertising. Based upon the NPI data 106, including other datasets obtained from data providers-brokers-sellers 20, impression data, and selected parameters and filters from a client system, the DSP campaign planning system 108 can be used to plan a distribution strategy of content from one of the client systems 112a, 112b,112n and to generate instructions for an advertising exchange server.

In an embodiment, DSP campaign planning system 108 is programmed to transmit data representing an audience, which has been defined using the system 108, to DSP 110. DSP 110 is programmed to transmit instructions and content to the advertising exchange system 120, which is programmed to select distribution channels to deliver the content as impressions such as through a media server 130 and a media and advertisement display channel 140. Example channels can include email, text messaging, websites that are viewed using a user computer 150 or other media. In embodiments, the advertising exchange system 120 can submit bids on behalf of a client system to deliver the content through selective channels based upon the distribution plan of such client system. Such channels may be identified to and selected by the DSP campaign planning system 108 based upon configuration provided in separate processes that are not germane to the focus of this disclosure. Channels can be selected or specified based upon bid amounts, cost, user behavior or goals of a campaign.

The elements of the distributed computer system as seen in FIG. 1 can be programmed to interoperate to implement an efficient and precisely targeted process of communicating content to user computer 150. Assume that user computer 150 is associated with a physician having NPI N who is browsing a website represented by display 140. The website is associated with the brand owner of medication M1, and as part of establishing an account with that website the physician has previously provided N to the website. An entity that owns or operates DSP system 110 and/or DSP campaign planning system 108 may communicate with operators of websites to program those websites to create and store a cookie associated with the entity on user computer 150. In some cases, the entity establishes contract partnerships with data brokers and/or data onboarders to aggregate and/or identify data to tie to devices of HCPs. During a browsing session, the website sets cookie 152 on user computer 150, and the cookie includes an encrypted or encoded copy of N, or a reference back to the website 140 from which N can be retrieved by authorized systems. This process essentially creates a local record at user computer 150 that N visited M1.

Now assume that the same physician browses to a different website unaffiliated with M1 that hosts digital advertisements that the different website obtains via media server 130 and advertising exchange 120. Both media server 130 and advertising exchange 120 are programmed to read cookie 152 if needed. When the physician arrives at the different website with a browser, the browser queries media server 130 for which advertisement to display, and media server passes the request to advertising exchange 120. Request transmissions in this sequence may include the contents of cookie 152 including the coded value of N. Advertising exchange 120 inspects bid data previously received from DSP system 110 and determines that a different medication brand, M2, is the high bidder for delivery of ads to physicians who have written more M2 prescriptions than for M1 in the past month. Furthermore, the bid data may include Nor a coded version of it, so that advertising exchange 120 can also determine that physician N is a qualified recipient of ads for M2 based on the bid and having previously read cookie 152.

Alternatively, advertising exchange 120 is programmed to query DSP system 110 to request whether N matches the criteria established in a particular bid for delivery of a particular advertisement. DSP campaign planning system 108 may be programmed to match N to claims data 102 and prescription data 104 to determine what procedure codes or prescriptions are associated with N and then determine whether a match exists. Presuming these checks are positive, an advertisement for M2 is then transmitted from advertising exchange 120 and media server 130 to the website for display at user computer 150.

In this manner, the system of FIG. 1 facilitates selecting digital data, such as for advertisements or offers, based upon combinations of data that have not been previously available, namely the intersection of claims data 102, prescription data 104, NPI data 106, demographic data 30, practice/specialty data 40, address data 50 and education data 60 in combination with the instructions and content provided under the distribution plan of DSP campaign planning system 108 and rules or bids established with DSP system 110, via media server 130 and advertising exchange 120, specifying which content to provide when values of the data match in different ways. Using these combination and matching techniques, content can be more efficiently routed to user computer 150 using fewer roundtrip network messages, less storage and fewer CPU cycles than otherwise would be required to determine how to show M2, in the foregoing example, to the user computer with other techniques. In this manner, the techniques herein provide a distinct technical solution to the technical problem of how to join disparate datasets to support selection of target audience for a particular user account or user.

### 2. Functional Overview

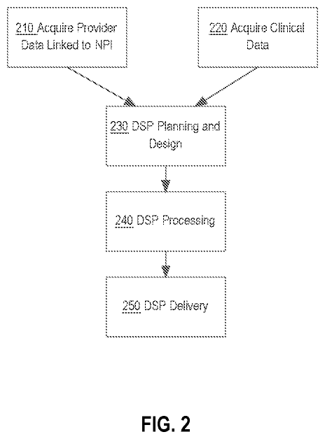

FIG. 2 illustrates a DSP process according to embodiments. In an embodiment, the implementation of the functions described herein using one or more computer programs or other software elements that are loaded into and executed using one or more general-purpose computers will cause the general-purpose computers to be configured as a particular machine or as a computer that is specially adapted to perform the functions described herein. Further, each of the flow diagrams that are described further herein may serve, alone or in combination with the descriptions of processes and functions in prose herein, as algorithms, plans or directions that may be used to program a computer or logic to implement the functions that are described. In other words, all the prose text herein, and all the drawing figures, together are intended to provide disclosure of algorithms, plans or directions that are sufficient to permit a skilled person to program a computer to perform the functions that are described herein, in combination with the skill and knowledge of such a person given the level of skill that is appropriate for inventions and disclosures of this type. Reference numerals in FIG. 2 indicate functional operations that can be implemented in one or more instructions of a computer programming source language and then compiled into one or more executables that are operable to provide the functions described for FIG. 2.

At operation 210, data linked to NPIs is obtained and delivered to a DSP campaign planning system at operation 230. The data obtained and delivered at operation 210 may include any of demographic data 30, practice/specialty data 40, address data 50, and education data 60 of FIG. 1. In an embodiment, clinical data is delivered to the DSP campaign planning system at operation 220. The further clinical data typically is associated with the practice areas of the NPIs and can consist of the claims data 102 and prescription data 104 of FIG. 1. In this manner, in some embodiments, all of claims data 102, prescription data 104, National Provider Identifier (NPI) data 106, demographic data 30, practice/specialty data 40, address data 50, and education data 60 are obtained from data providers-brokers-sellers 20 and persisted to shared database 107. In an embodiment, an application programming interface (API) can be utilized to obtain NPI-linked data.

At operation 230, based on NPI and clinical data received at operation 210 and operation 220, DSP system 230 can be utilized by a client system to configure planning parameters for delivering content to targeted HCPs as impressions such as further described herein. Once parameters of the planning strategy have been configured, the DSP system can process and generate a distribution plan at operation 240.

Once the distribution plan has been generated, the plan can be implemented or delivered at operation 250. Delivery may comprise of delivering particular content to an advertising exchange server for distribution through any available media channels.

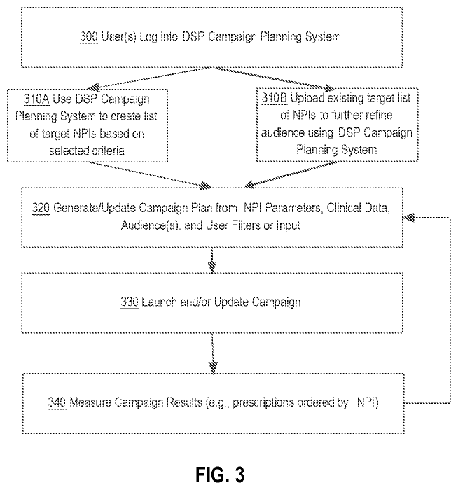

FIG. 3 is a flow diagram of a DSP plan configuration and implementation process according to embodiments.

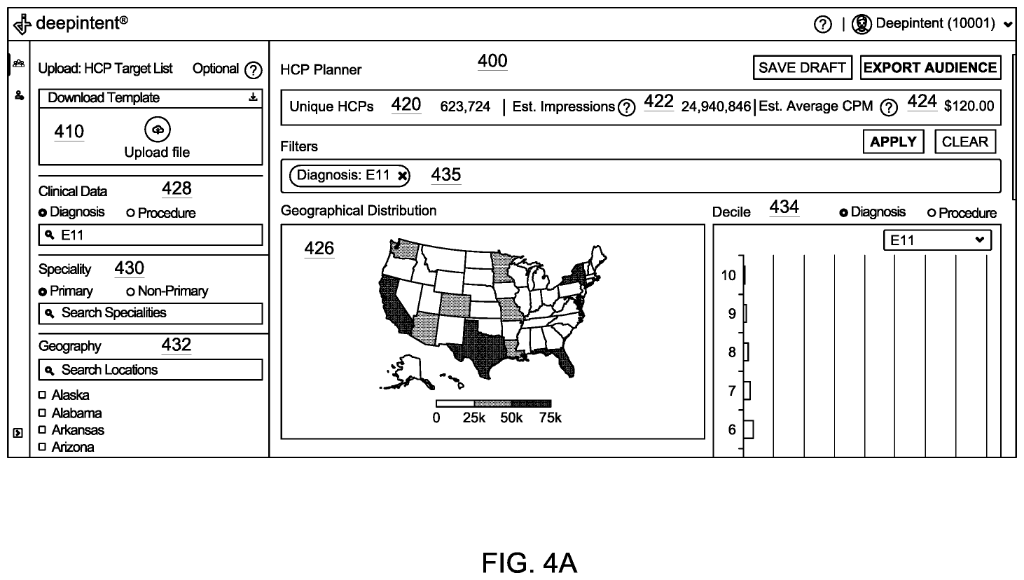

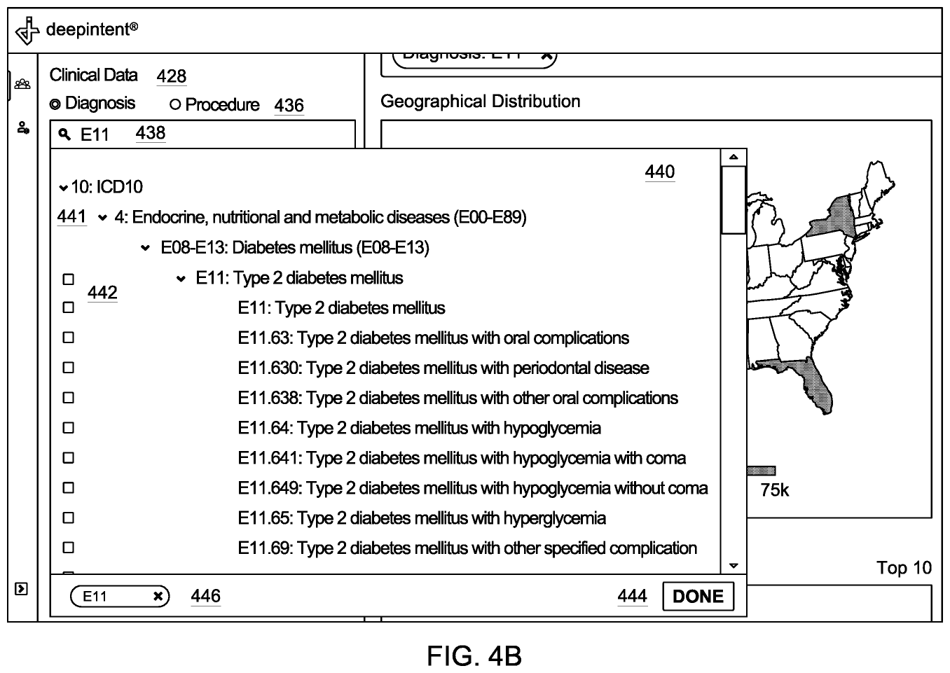

At operation 300, a client system can begin the process of generating a distribution plan by utilizing a DSP planning tool such as DSP campaign planning system 108 of FIG. 1. In an embodiment, with DSP campaign planning system 108, a set of NPIs forming a starting point for defining a target audience can be supplied by uploading an existing list, or by creating a list, as represented in operation 310A, 310B. In various embodiments, at step 310A the DSP campaign planning system 108 may be used to create a list of target NPIs based on selected criteria. Additionally or alternatively, at step 310B the DSP campaign planning system 108 may be used to upload an existing target list of NPIs to further refine an audience using the planning system. For example, in one embodiment a client, user or user account may have access to an existing NPI list and may upload that list to the DSP campaign planning system 108, then continue to filter the list to create an optimal audience for that campaign using filter tools that are described in other sections herein. Or, no list may be provided and the DSP campaign planning system 108 may be used to create an optimal audience using the filter tools. In either case, a display showing the list can include the NPI practice area(s), prescription history, medical claim history, procedure history, other clinical data, and/or geography and then filtered using tools as shown, for example, in FIG. 4A and subsequent drawing figures. The NPI related data and display can be filtered and narrowed, for example, to particular practice areas, medical procedures, codes, etc., so as to represent particular audiences for purposes of campaign planning.

Based upon parameters and filters selected using the planning tool, a distribution/campaign plan is generated by the DSP at operation 320.

At operation 330, the campaign is launched such as by sending instructions to an advertising exchange server for distributing campaign content such as advertisements to various media channels for impressions intended to reach appropriate HCPs.

At operation 340, results of the launched campaign are measured such as by tracking impressions delivered from various media channels, feedback in response to the impressions, and/or other measured results.

The tracked results can be fed back to the DSP system and/or campaign clients/agents at operation 340, from which the DSP system and planning tool can be used to adjust the campaign/planning instructions at operation 320 in order to relaunch or update the campaign at operation 330. In embodiments, campaign updates and relaunches can be configured in the DSP system to perform automatically or require client user input.

FIG. 4A illustrates a computer-generated graphical user interface (GUI) of a DSP campaign planning system according to an embodiment. In one embodiment, DSP campaign planning system 108 generates the GUI of FIG. 4A via instructions, such as dynamic HTML, which may be rendered using a compatible browser that is executed at client 112a to produce a visual display on a computer display device corresponding to FIG. 4A. This same form of generating instructions for rendering at a client may be programmed at DSP campaign planning system 108 for all the GUI examples of this disclosure.

In an embodiment, a GUI 400 of FIG. 4A comprises a plurality of display panels, widgets, and graphical menus or tools that are programmed to execute as next described, to enable users of DSP campaign planning system 108 to select parameters and preview campaign projections during a process of preparing a strategy and distributing a clinically relevant HCP audience to the DSP. At link 410, a user can select to open and/or upload an HCP target list which will designate the HCPs to which the campaign is targeted. For example, an HCP target list can be uploaded from a stored file, retrieved from a previously uploaded file and/or entered manually in various embodiments. Field 420 indicates the number of unique HCPs available to be digitally targeted. Field 422 indicates the estimated number of impressions available to the campaign based upon the selected target HCPs and DSP parameters. Field 424 indicates the estimated cost per thousand impressions (CPM) to the client based upon the current campaign configuration and may include media cost as well as data cost. In an embodiment, DSP campaign planning system 108 is programmed, in response to user input indicating hovering over a geographical distribution map 426, to generate a window summarizing the number of unique HCPs targeted in the geographic area and number of estimated impressions available in the selected geographic area. Additionally or alternatively, user input may specify values in a clinical data panel 428, specialty panel 430 or geography panel 432 to further filter and refine a target list of NPIs. Clinical data panel 428 may be used to specify a diagnosis code or procedure code of HCPs to which impressions should be targeted. Specialty panel 430 may be used to specify a primary or non-primary specialty of HCPs to which impressions should be targeted. Geography panel 432 may be used to select one or more geographical regions, including but not limited to countries, states, counties or other regions in various embodiments, of HCPs to which impressions should be targeted. In some embodiments, ZIP code, DMA or other values may be used to filter and define audiences. Furthermore, for purposes of illustrating a clear example, this disclosure focuses on NPI as a practitioner identifier relevant to the United States, embodiments for other territories could be based on any identifier that a particular country uses to uniquely identify HCPs; examples include the MINC identifier for Canada, Practitioner Identifier for UK, etc. FIG. 4A illustrates, for purposes of a clear example, an embodiment focused on the United States and states of the United States, but other embodiments may be implemented for any other geographic regions represented in HCP data that can be obtained from data providers.

Thus, to focus the campaign to particular HCPs, filter parameters at panels 428, 430, 432 can be selected including, for example, diagnosis and procedure codes (e.g., ICD-10 or CPT codes), practice specialty, and practice geography associated with the HCPs to be targeted. In an embodiment, DSP campaign planning system 108 is programmed to display a decile panel 434 comprising a histogram or other plurality of bars representing particular subgroups of HCPs to be targeted. For purposes of illustrating a clear example, FIG. 4A shows an embodiment in which ten (10) deciles are displayed but other embodiments may interoperate with different subgroups such as percentiles. The organization of HCP data into deciles or other subgroups may be programmed as part of DSP campaign planning system 108. Additionally or alternatively, an entity that owns or operates DSP campaign planning system 108 may calculate the deciles or decile associations may be indicated in metadata of HCP data in the form that is originally received from data providers. In an embodiment, in response to user input indicating a selection of one of the deciles in decile panel 434, DSP campaign planning system 108 is programmed to further filter a campaign audience based on the selected deciles. The DSP campaign planning system 108 also may be programmed to allow custom filters to be configured by the user computer to further target particular groups of HCPs.

FIG. 4B illustrates a GUI that is programmed to support selecting DSP filter parameters in one embodiment. FIG. 4B represents an expanded display of detailed UI elements for clinical data panel 428 of FIG. 4A; in other embodiments, DSP campaign planning system 108 is programmed, in response to input selecting specialty panel 430 or geography panel 432, to generate displays similar to FIG. 4B to accept input of detailed filter parameters relating to specialty and/or geography. In an embodiment, DSP campaign planning system 108 is programmed to display clinical data panel 428 with radio buttons 436; in an embodiment, the radio buttons are programmed to accept input specifying a diagnosis or procedure. DSP campaign planning system 108 is further programmed to display an input box 438 in which a diagnosis code or procedure code may be entered, respectively, depending on which radio button 436 is selected. In the example of FIG. 4B, Diagnosis radio button 436 is selected and code “E11” has been entered in input box 438.

In response to this input box 438, DSP campaign planning system 108 is programmed to search for particular parameters in a hierarchical ontology of filter terms that match the input at input box 438. For example, a user computer can enter a particular diagnosis code or procedure code in input box 438, and results of a search are displayed in panel 440 showing a hierarchical tree 441 of codes and code definitions most closely matching the search expression that was entered in the input box. The tree 441 can list standard descriptions of codes or other metadata associated with codes. Tree 441 may comprise a plurality of expansion icons 442 which, when selected, cause DSP campaign planning system 108 to update the tree to further expand or narrow the contents of the tree. In this manner, a user computer can interact with tree 441 in multiple input operations to specify HCPs associated with target codes at any level of granularity. In an embodiment, as user input is received to select codes for filtering, a filter list 442 is updated to display all then currently selected filter terms to provide reinforcement to the user of the scope of filtering that will be performed. Selecting a Done button 444 closes window 440 and causes updating a filter list 435 (FIG. 4A) to indicate all filters that were selected, while concurrently storing the specified filter list for later use in delivering a planned campaign.

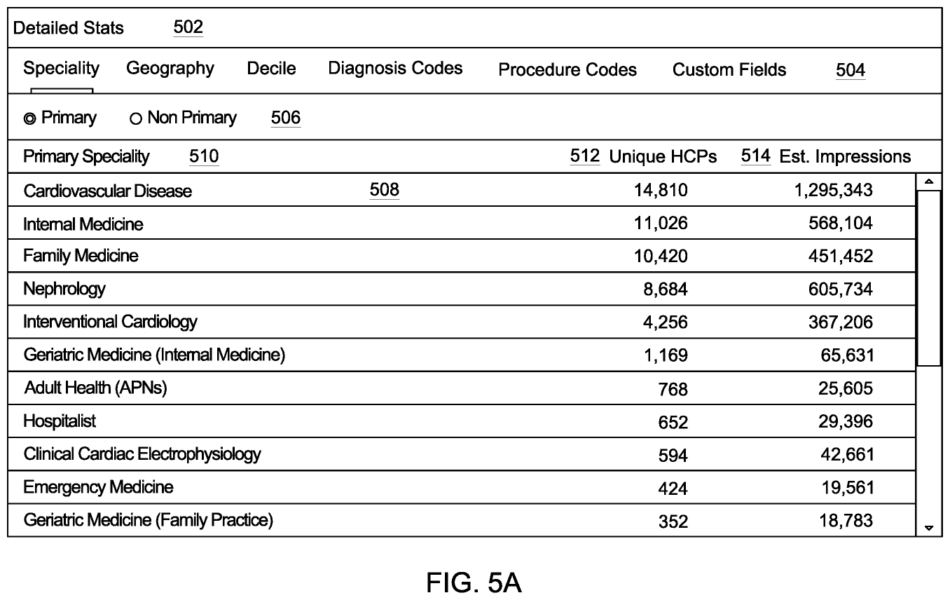

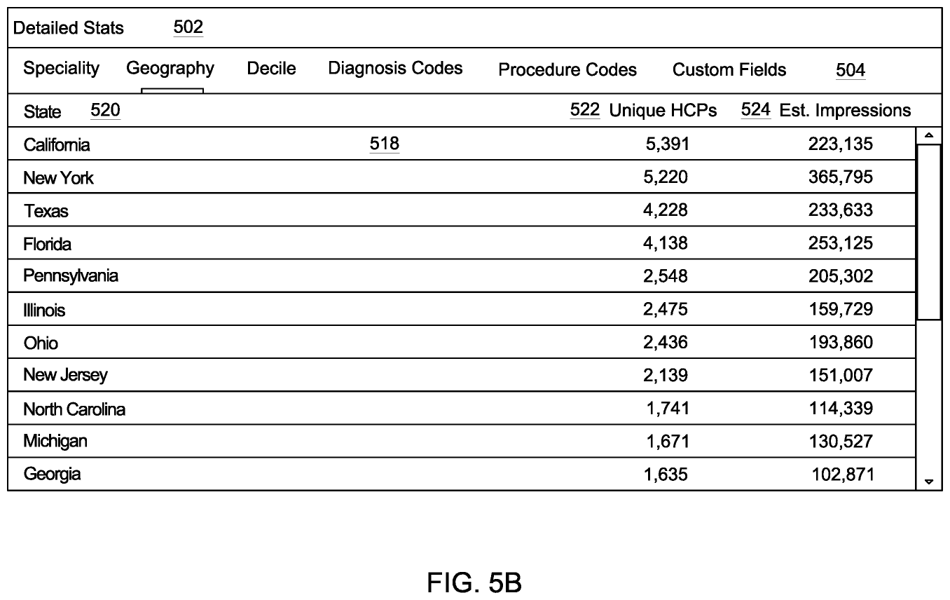

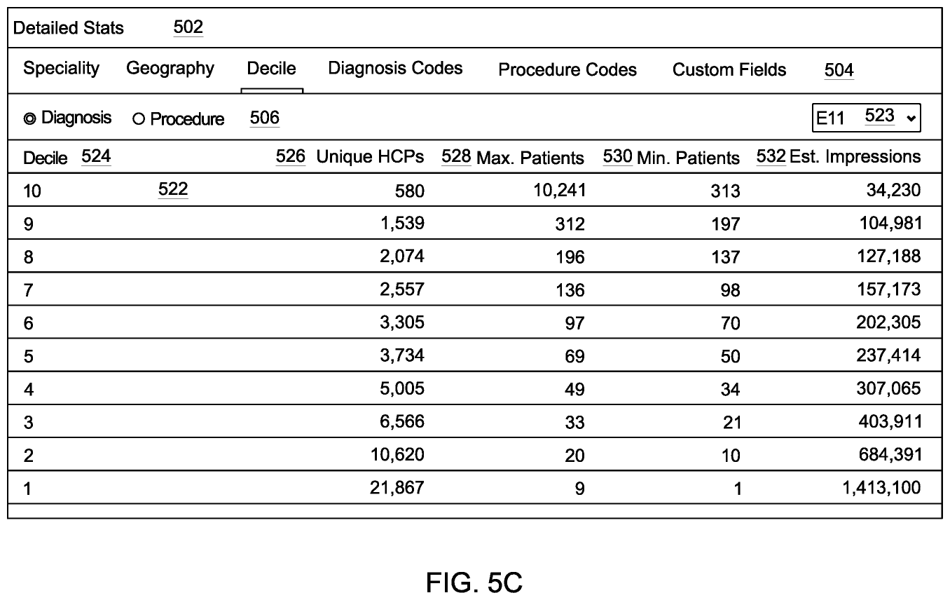

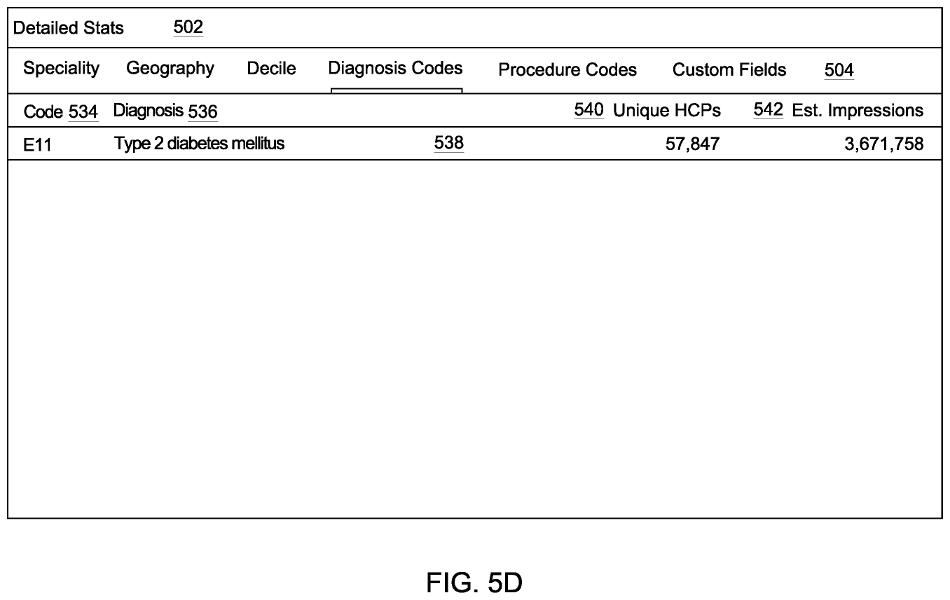

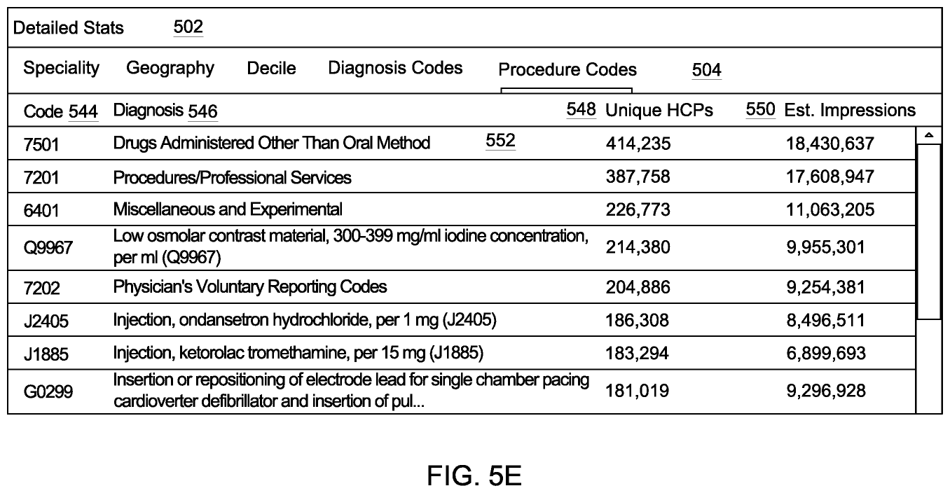

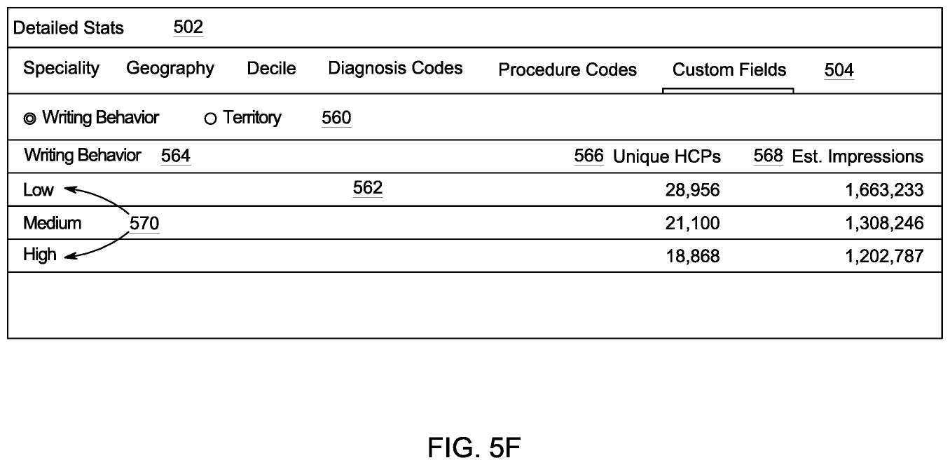

FIG. 5A is a GUI display for reviewing DSP planning data of HCP specialties according to embodiments. In an embodiment, in response to input from a user computer requesting statistical data for HCPs, DSP campaign planning system 108 is programmed to display a detailed stats window 502. In various embodiments, the statistical data shown in window 502 may be derived from unfiltered HCP data stored in shared database 107, or for filtered HCP data that has been specified via the processes of FIG. 4A, FIG. 4B.

In an embodiment, window 502 comprises a category list 504 that is programmed to accept input specifying a category of detailed statistics to display. In an embodiment, the category “Specialty” is displayed, and Geography, Decile, Diagnosis Codes, Procedure Codes and Custom Fields also may be displayed; other embodiments may be programmed to address more or fewer categories and FIG. 5A is merely an example of possible categories.

In an embodiment, window 502 further comprises radio buttons 506 that are programmed to receive input to further refine the statistical data that is displayed. The number and identity of radio buttons 506 are context-sensitive and are programmed to change based upon which category is selected in category list 504. In the example of FIG. 5A, radio buttons 506 comprise Primary or Non-Primary as these relate to Specialty.

In response to selection of options in category list 504 and radio buttons 506, in an embodiment, DSP campaign planning system 108 is programmed to display a statistical table 508 of statistical data matching the selected options. In the example of FIG. 5A, table 508 lists and compares HCP specialties relative to numbers of unique HCPs targeted, and estimated impressions available based upon the currently configured campaign plan. In an embodiment, DSP campaign planning system 108 may be programmed to display unique numbers of patients in table 508. In an embodiment, column headers 510, 512, 514 identify data in columns below the headings and are programmed as sortable headings; user computer input selecting one of the column headers causes DSP campaign planning system 108 to sort the table 508 in inverse order and update the display of FIG. 5A to show data in a changed sort order.

FIG. 5B is a GUI display for reviewing DSP planning data of HCP geography according to embodiments. FIG. 5B shows window 502 of FIG. 5A in which the category Geography has been selected via user computer input in category list 504. In response, DSP campaign planning system 108 is programmed to display a table 518 listing and comparing HCP geographic locations, as indicated by column header 520, relative to numbers of unique HCPs targeted and estimated impressions produced by the currently configured campaign as indicated by column headers 522, 524 respectively. Column headers 520, 522, 524 may be programmed to support sorting and redisplaying data of table 518 in response to user computer input selecting a column header, as described above.

FIG. 5C is a GUI display for reviewing target HCP data by decile grouping according to embodiments. FIG. 5C shows window 502 of FIG. 5A in which the category Decile has been selected via user computer input in category list 504. In response, DSP campaign planning system 108 is programmed to display radio buttons 506, which are programmed to receive user computer input specifying a diagnosis or procedure. In response to input at both category list 504 and buttons 506, DSP campaign planning system 108 is programmed to display table 522 showing data organized by decile as indicated by column header 524. In an embodiment, each decile represents a subgroup of a procedure code or an ICD-10 Code group such as category E11, which refers to Type 2 diabetes mellitus, as indicated by widget 523. For each decile, a count of unique HCPs, maximum and minimum estimated number of patients for each HCP per decile, and an estimated number of impressions that are available for the HCPs per decile are listed in table 522 as indicated by column headers 526, 528, 530, 532. Column headers 526, 528, 530, 532 may be programmed to support sorting and redisplaying data of table 522 in response to user computer input selecting a column header, as described above. Different ICD-10 codes can be selected for review by utilizing a drop-down menu of widget 523.

FIG. 5D is a GUI display for reviewing HCP clinical/diagnosis code data for DSP planning according to embodiments. FIG. 5D shows window 502 of FIG. 5A in which the category Diagnosis Codes has been selected via user computer input in category list 504. In response, DSP campaign planning system 108 is programmed to display a table listing the one or more diagnosis codes that have been previously selected via the filtering options discussed in previous sections above, a diagnosis description for a corresponding code, the number of unique HCPs associated with the diagnosis code(s), and estimated number of available impressions targeting the associated unique HCPs who are associated with those diagnosis code(s), as indicated by column headers 534, 536, 540, 542. For purposes of illustrating a clear example, FIG. 5D shows a single result row of data but for other filter configurations, values or embodiments any number of rows may be displayed in a table as seen in FIG. 5D. Column headers 534, 536, 540, 542 may be programmed to support sorting and redisplaying data of table 538 in response to user computer input selecting a column header, as described above.

FIG. 5E is a GUI display illustrating top procedures of HCPs that have been pre-populated based upon the HCP clinical/procedure code that was previously selected in FIG. 5D. FIG. 5E shows window 502 of FIG. 5A in which the category Procedure Codes has been selected via user computer input in category list 504. In response, DSP campaign planning system 108 is programmed to display a table 552 listing procedure codes associated with targeted HCPs, names of procedures corresponding to procedure codes, the number of unique HCPs associated with the procedure codes, and estimated number of available impressions targeting the associated unique HCPs, as indicated by column headers 544, 546, 548, 550 respectively. Column headers 544, 546, 548, 550 may be programmed to support sorting and redisplaying data of table 552 in response to user computer input selecting a column header, as described above.

FIG. 5F is a GUI display for showing targeted HCP data based upon one or more custom fields. FIG. 5F shows window 502 of FIG. 5A in which the category Custom Fields has been previously uploaded by the client within the NPI target list, and user computer input has selected the category “Custom Fields” within category list 504 to cause a display of custom field data. In response, DSP campaign planning system 108 is programmed to display one or more radio buttons 560 that are programmed to accept user computer input selecting one custom field from among a plurality of custom fields that have been previously programmed and that are represented in HCP data in shared database 107. In the example of FIG. 5F, two custom fields have been programmed and are denoted Writing Behavior and Territory. Writing Behavior may refer to the general magnitude of prescription writing by HCPs, for example, as indicated in prescription data 104 (FIG. 1) and Territory may refer to a broad geographical region. In response, DSP campaign planning system 108 is programmed to display a table 562 listing custom field values associated with targeted HCPs, the number of unique HCPs associated with the procedure codes, and estimated number of available impressions targeting the associated unique HCPs, as indicated by column headers 564, 566, 568 respectively. In an embodiment, the specific values 570 displayed under the first column header 564 are dynamically updated in the display depending upon which radio button 560 is selected for a particular custom field. The example of FIG. 5F shows values of Low, Medium, High for custom field Writing Behavior, but if custom field Territory had been selected, different values would appear. Column headers 564, 566, 568 may be programmed to support sorting and redisplaying data of table 552 in response to user computer input selecting a column header, as described above.

### 3. Example Hardware Components

According various embodiments, the techniques described herein are implemented by at least one computing device. The techniques may be implemented in whole or in part using a combination of at least one server computer and/or other computing devices that are coupled using a network, such as a packet data network. The computing devices may be hard-wired to perform the techniques, or may include digital electronic devices such as at least one application-specific integrated circuit (ASIC) or field programmable gate array (FPGA) that is persistently programmed to perform the techniques, or may include at least one general purpose hardware processor programmed to perform the techniques pursuant to program instructions in firmware, memory, other storage, or a combination. Such computing devices may also combine custom hard-wired logic, ASICs, or FPGAs with custom programming to accomplish the described techniques. The computing devices may be server computers, workstations, personal computers, portable computer systems, handheld devices, mobile computing devices, wearable devices, body mounted or implantable devices, smartphones, smart appliances, internetworking devices, autonomous or semi-autonomous devices such as robots or unmanned ground or aerial vehicles, any other electronic device that incorporates hard-wired and/or program logic to implement the described techniques, one or more virtual computing machines or instances in a data center, and/or a network of server computers and/or personal computers.

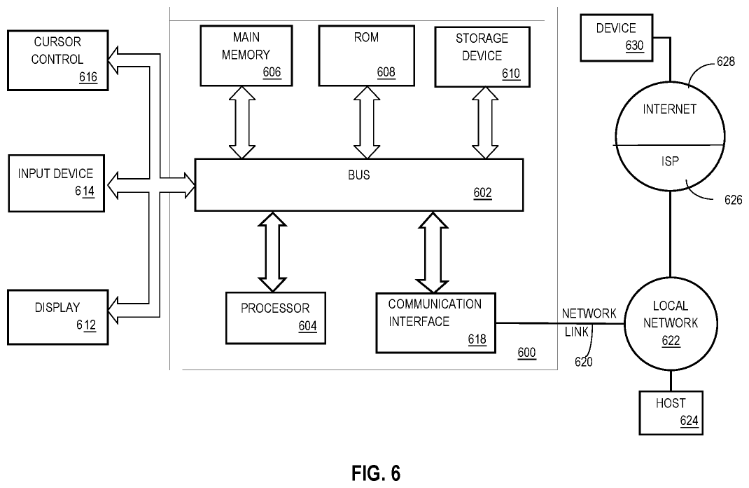

FIG. 6 is a block diagram that illustrates an example computer system with which an embodiment may be implemented. In the example of FIG. 6, a computer system 600 and instructions for implementing the disclosed technologies in hardware, software, or a combination of hardware and software, are represented schematically, for example as boxes and circles, at the same level of detail that is commonly used by persons of ordinary skill in the art to which this disclosure pertains for communicating about computer architecture and computer systems implementations.

Computer system 600 includes an input/output (I/O) subsystem 602 which may include a bus and/or other communication mechanism(s) for communicating information and/or instructions between the components of the computer system 600 over electronic signal paths. The I/O subsystem 602 may include an I/O controller, a memory controller and at least one I/O port. The electronic signal paths are represented schematically in the drawings, for example as lines, unidirectional arrows, or bidirectional arrows.

At least one hardware processor 604 is coupled to I/O subsystem 602 for processing information and instructions. Hardware processor 604 may include, for example, a general-purpose microprocessor or microcontroller and/or a special-purpose microprocessor such as an embedded system or a graphics processing unit (GPU) or a digital signal processor or ARM processor. Processor 604 may comprise an integrated arithmetic logic unit (ALU) or may be coupled to a separate ALU.

Computer system 600 includes one or more units of memory 606, such as a main memory, which is coupled to I/O subsystem 602 for electronically digitally storing data and instructions to be executed by processor 604. Memory 606 may include volatile memory such as various forms of random-access memory (RAM) or other dynamic storage device. Memory 606 also may be used for storing temporary variables or other intermediate information during execution of instructions to be executed by processor 604. Such instructions, when stored in non-transitory computer-readable storage media accessible to processor 604, can render computer system 600 into a special-purpose machine that is customized to perform the operations specified in the instructions.

Computer system 600 further includes non-volatile memory such as read only memory (ROM) 608 or other static storage device coupled to I/O subsystem 602 for storing information and instructions for processor 604. The ROM 608 may include various forms of programmable ROM (PROM) such as erasable PROM (EPROM) or electrically erasable PROM (EEPROM). A unit of persistent storage 610 may include various forms of non-volatile RAM (NVRAM), such as FLASH memory, or solid-state storage, magnetic disk or optical disk such as CD-ROM or DVD-ROM, and may be coupled to I/O subsystem 602 for storing information and instructions. Storage 610 is an example of a non-transitory computer-readable medium that may be used to store instructions and data which when executed by the processor 604 cause performing computer-implemented methods to execute the techniques herein.

The instructions in memory 606, ROM 608 or storage 610 may comprise one or more sets of instructions that are organized as modules, methods, objects, functions, routines, or calls. The instructions may be organized as one or more computer programs, operating system services, or application programs including mobile apps. The instructions may comprise an operating system and/or system software; one or more libraries to support multimedia, programming or other functions; data protocol instructions or stacks to implement TCP/IP, HTTP or other communication protocols; file processing instructions to interpret and render files coded using HTML, XML, JPEG, MPEG or PNG; user interface instructions to render or interpret commands for a graphical user interface (GUI), command-line interface or text user interface; application software such as an office suite, internet access applications, design and manufacturing applications, graphics applications, audio applications, software engineering applications, educational applications, games or miscellaneous applications. The instructions may implement a web server, web application server or web client. The instructions may be organized as a presentation layer, application layer and data storage layer such as a relational database system using structured query language (SQL) or no SQL, an object store, a graph database, a flat file system or other data storage.

Computer system 600 may be coupled via I/O subsystem 602 to at least one output device 612. In one embodiment, output device 612 is a digital computer display. Examples of a display that may be used in various embodiments include a touch screen display or a light-emitting diode (LED) display or a liquid crystal display (LCD) or an e-paper display. Computer system 600 may include other type(s) of output devices 612, alternatively or in addition to a display device. Examples of other output devices 612 include printers, ticket printers, plotters, projectors, sound cards or video cards, speakers, buzzers or piezoelectric devices or other audible devices, lamps or LED or LCD indicators, haptic devices, actuators or servos.

At least one input device 614 is coupled to I/O subsystem 602 for communicating signals, data, command selections or gestures to processor 604. Examples of input devices 614 include touch screens, microphones, still and video digital cameras, alphanumeric and other keys, keypads, keyboards, graphics tablets, image scanners, joysticks, clocks, switches, buttons, dials, slides, and/or various types of sensors such as force sensors, motion sensors, heat sensors, accelerometers, gyroscopes, and inertial measurement unit (IMU) sensors and/or various types of transceivers such as wireless, such as cellular or Wi-Fi, radio frequency (RF) or infrared (IR) transceivers and Global Positioning System (GPS) transceivers.

Another type of input device is a control device 616, which may perform cursor control or other automated control functions such as navigation in a graphical interface on a display screen, alternatively or in addition to input functions. Control device 616 may be a touchpad, a mouse, a trackball, or cursor direction keys for communicating direction information and command selections to processor 604 and for controlling cursor movement on display 612. The input device may have at least two degrees of freedom in two axes, a first axis (e.g., x) and a second axis (e.g., y), that allows the device to specify positions in a plane. Another type of input device is a wired, wireless, or optical control device such as a joystick, wand, console, steering wheel, pedal, gearshift mechanism or other type of control device. An input device 614 may include a combination of multiple different input devices, such as a video camera and a depth sensor.

In another embodiment, computer system 600 may comprise an internet of things (IoT) device in which one or more of the output device 612, input device 614, and control device 616 are omitted. Or, in such an embodiment, the input device 614 may comprise one or more cameras, motion detectors, thermometers, microphones, seismic detectors, other sensors or detectors, measurement devices or encoders and the output device 612 may comprise a special-purpose display such as a single-line LED or LCD display, one or more indicators, a display panel, a meter, a valve, a solenoid, an actuator or a servo.

When computer system 600 is a mobile computing device, input device 614 may comprise a global positioning system (GPS) receiver coupled to a GPS module that is capable of triangulating to a plurality of GPS satellites, determining and generating geo-location or position data such as latitude-longitude values for a geophysical location of the computer system 600. Output device 612 may include hardware, software, firmware and interfaces for generating position reporting packets, notifications, pulse or heartbeat signals, or other recurring data transmissions that specify a position of the computer system 600, alone or in combination with other application-specific data, directed toward host 624 or server 630.

Computer system 600 may implement the techniques described herein using customized hard-wired logic, at least one ASIC or FPGA, firmware and/or program instructions or logic which when loaded and used or executed in combination with the computer system causes or programs the computer system to operate as a special-purpose machine. According to one embodiment, the techniques herein are performed by computer system 600 in response to processor 604 executing at least one sequence of at least one instruction contained in main memory 606. Such instructions may be read into main memory 606 from another storage medium, such as storage 610. Execution of the sequences of instructions contained in main memory 606 causes processor 604 to perform the process steps described herein. In alternative embodiments, hard-wired circuitry may be used in place of or in combination with software instructions.

The term “storage media” as used herein refers to any non-transitory media that store data and/or instructions that cause a machine to operation in a specific fashion. Such storage media may comprise non-volatile media and/or volatile media. Non-volatile media includes, for example, optical or magnetic disks, such as storage 610. Volatile media includes dynamic memory, such as memory 606. Common forms of storage media include, for example, a hard disk, solid state drive, flash drive, magnetic data storage medium, any optical or physical data storage medium, memory chip, or the like.

Storage media is distinct from but may be used in conjunction with transmission media. Transmission media participates in transferring information between storage media. For example, transmission media includes coaxial cables, copper wire and fiber optics, including the wires that comprise a bus of I/O subsystem 602. Transmission media can also take the form of acoustic or light waves, such as those generated during radio-wave and infra-red data communications.

Various forms of media may be involved in carrying at least one sequence of at least one instruction to processor 604 for execution. For example, the instructions may initially be carried on a magnetic disk or solid-state drive of a remote computer. The remote computer can load the instructions into its dynamic memory and send the instructions over a communication link such as a fiber optic or coaxial cable or telephone line using a modem. A modem or router local to computer system 600 can receive the data on the communication link and convert the data to be read by computer system 600. For instance, a receiver such as a radio frequency antenna or an infrared detector can receive the data carried in a wireless or optical signal and appropriate circuitry can provide the data to I/O subsystem 602 such as place the data on a bus. I/O subsystem 602 carries the data to memory 606, from which processor 604 retrieves and executes the instructions. The instructions received by memory 606 may optionally be stored on storage 610 either before or after execution by processor 604.

Computer system 600 also includes a communication interface 618 coupled to bus 602. Communication interface 618 provides a two-way data communication coupling to network link(s) 620 that are directly or indirectly connected to at least one communication networks, such as a network 622 or a public or private cloud on the Internet. For example, communication interface 618 may be an Ethernet networking interface, integrated-services digital network (ISDN) card, cable modem, satellite modem, or a modem to provide a data communication connection to a corresponding type of communications line, for example an Ethernet cable or a metal cable of any kind or a fiber-optic line or a telephone line. Network 622 broadly represents a local area network (LAN), wide-area network (WAN), campus network, internetwork or any combination thereof. Communication interface 618 may comprise a LAN card to provide a data communication connection to a compatible LAN, or a cellular radiotelephone interface that is wired to send or receive cellular data according to cellular radiotelephone wireless networking standards, or a satellite radio interface that is wired to send or receive digital data according to satellite wireless networking standards. In any such implementation, communication interface 618 sends and receives electrical, electromagnetic or optical signals over signal paths that carry digital data streams representing various types of information.

Network link 620 typically provides electrical, electromagnetic, or optical data communication directly or through at least one network to other data devices, using, for example, satellite, cellular, Wi-Fi, or BLUETOOTH technology. For example, network link 620 may provide a connection through a network 622 to a host computer 624.

Furthermore, network link 620 may provide a connection through network 622 or to other computing devices via internetworking devices and/or computers that are operated by an Internet Service Provider (ISP) 626. ISP 626 provides data communication services through a world-wide packet data communication network represented as internet 628. A server computer 630 may be coupled to internet 628. Server 630 broadly represents any computer, data center, virtual machine or virtual computing instance with or without a hypervisor, or computer executing a containerized program system such as DOCKER or KUBERNETES. Server 630 may represent an electronic digital service that is implemented using more than one computer or instance and that is accessed and used by transmitting web services requests, uniform resource locator (URL) strings with parameters in HTTP payloads, API calls, app services calls, or other service calls. Computer system 600 and server 630 may form elements of a distributed computing system that includes other computers, a processing cluster, server farm or other organization of computers that cooperate to perform tasks or execute applications or services. Server 630 may comprise one or more sets of instructions that are organized as modules, methods, objects, functions, routines, or calls. The instructions may be organized as one or more computer programs, operating system services, or application programs including mobile apps. The instructions may comprise an operating system and/or system software; one or more libraries to support multimedia, programming or other functions; data protocol instructions or stacks to implement TCP/IP, HTTP or other communication protocols; file format processing instructions to interpret or render files coded using HTML, XML, JPEG, MPEG or PNG; user interface instructions to render or interpret commands for a graphical user interface (GUI), command-line interface or text user interface; application software such as an office suite, internet access applications, design and manufacturing applications, graphics applications, audio applications, software engineering applications, educational applications, games or miscellaneous applications. Server 630 may comprise a web application server that hosts a presentation layer, application layer and data storage layer such as a relational database system using structured query language (SQL) or no SQL, an object store, a graph database, a flat file system or other data storage.

Computer system 600 can send messages and receive data and instructions, including program code, through the network(s), network link 620 and communication interface 618. In the Internet example, a server 630 might transmit a requested code for an application program through Internet 628, ISP 626, local network 622 and communication interface 618. The received code may be executed by processor 604 as it is received, and/or stored in storage 610, or other non-volatile storage for later execution.

The execution of instructions as described in this section may implement a process in the form of an instance of a computer program that is being executed, and consisting of program code and its current activity. Depending on the operating system (OS), a process may be made up of multiple threads of execution that execute instructions concurrently. In this context, a computer program is a passive collection of instructions, while a process may be the actual execution of those instructions. Several processes may be associated with the same program; for example, opening up several instances of the same program often means more than one process is being executed. Multitasking may be implemented to allow multiple processes to share processor 604. While each processor 604 or core of the processor executes a single task at a time, computer system 600 may be programmed to implement multitasking to allow each processor to switch between tasks that are being executed without having to wait for each task to finish. In an embodiment, switches may be performed when tasks perform input/output operations, when a task indicates that it can be switched, or on hardware interrupts. Time-sharing may be implemented to allow fast response for interactive user applications by rapidly performing context switches to provide the appearance of concurrent execution of multiple processes simultaneously. In an embodiment, for security and reliability, an operating system may prevent direct communication between independent processes, providing strictly mediated and controlled inter-process communication functionality.

### 4. Extensions and Alternatives

In the foregoing specification, embodiments of the disclosure have been described with reference to numerous specific details that may vary from implementation to implementation. The specification and drawings are, accordingly, to be regarded in an illustrative rather than a restrictive sense. The sole and exclusive indicator of the scope of the disclosure, and what is intended by the applicants to be the scope of the disclosure, is the literal and equivalent scope of the set of claims that issue from this application, in the specific form in which such claims issue, including any subsequent correction

What is claimed is:

1. A computer implemented method for transmitting targeted digital impressions to a filtered audience, the method comprising:
receiving, using one or more computers programmed to implement a demand side platform (DSP) campaign planning system, a first list of one or more healthcare provider identifiers each uniquely identifying healthcare providers;
receiving, at the one or more computers, practice data comprising one or more of: clinical medical data indexed by the healthcare provider identifiers, prescription data specifying drug prescriptions previously written and indexed by the healthcare provider identifiers and/or medical claims data indexed by the healthcare provider identifiers and storing the data in a shared database;
displaying, in a graphical user interface that is generated by the DSP campaign planning system and provided to one or more client computers, counts of healthcare providers based upon using the first list and matching healthcare provider identifiers in the first list to the practice data;
receiving, from one or more client computers, input specifying one or more filter attributes, and in response, filtering the first list based on the one or more filter attributes to produce a filtered second list representing a target audience of healthcare provider identifiers;
transmitting the filtered second list of healthcare provider identifiers to a DSP service implemented using one or more DSP server computers, the DSP service being programmed to receive a particular healthcare provider identifier, match the particular healthcare provider identifier to the filtered second list, and in response to a match, generate instructions for use by one or more of an advertising exchange, media server and/or media and advertisement display channel to cause presentation of a targeted digital impression on a user computer associated with the particular healthcare provider identifier.

2. The method of claim 1, the healthcare provider identifiers comprising any of United States National Provider identifiers (NPIs), CINC values or Practitioner Identifiers.

3. The method of claim 1, further comprising receiving, at the one or more computers, for use in association with the practice data, one or more of demographic data, specialty data, best-of-breed address data and/or education data, each being indexed by the healthcare provider identifiers.

4. The method of claim 1 wherein the practice data comprises at least one of diagnosis, procedure, or prescription history associated with the healthcare provider identifiers.

5. The method of claim 1 wherein the clinical medical data comprises practice and patient statistics related to at least one of HCP specialty, geography, or HCP claim codes.

6. The method of claim 1, further comprising repeating, two or more times, the steps of displaying in a graphical user interface that is generated by the DSP campaign planning system and receiving input specifying one or more filter attributes, thereby repeatedly updating the filtered second list according to different filter attributes.

7. The method of claim 1 further comprising generating and displaying a graphical user interface that is programmed to receive client computer input specifying filter attributes for clinical data by diagnosis or procedure, for specialty by primary or non-primary specialty, for geography, for deciles of the healthcare provider identifiers, for diagnosis codes, and for procedure codes.

8. The method of claim 1, the DSP service being programmed for:
generating instructions for ranking impressions based upon one or more of target HCP practice specialties, target HCP geography, target HCP procedure codes, counts of unique HCPs, counts of unique HCP patients, or estimated numbers of impressions;
generating instructions for submitting bids for purchasing impressions based upon the ranking of the impressions.

9. The method of claim 1, the DSP service being programmed for:
generating instructions for ranking impressions based upon one or more of target HCP practice specialties, target HCP geography, target HCP procedure codes, counts of unique HCPs, counts of unique HCP patients, or estimated numbers of impressions;
generating instructions for submitting bids for purchasing impressions based upon the ranking of the impressions;
the method further comprising:
receiving, across a computer network, results of impressions that were generated in response to the digital publicizing instructions;
based upon the results of impressions, revising instructions for purchasing impressions;
transmitting the revised instructions to an advertising exchange server for executing one or more targeted digital impressions.

10. The method of claim 1, the particular healthcare provider identifier being received from a cookie, mobile advertising identifier, third-party identity token, or other digital identity data value that is capable of use in digital advertising.

11. One or more non-transitory computer readable storage media instructions which when executed using one or more computers cause the one or more computers to perform:
receiving, using one or more computers programmed to implement a demand side platform (DSP) campaign planning system, a first list of one or more healthcare provider identifiers each uniquely identifying healthcare providers;
receiving, at the one or more computers, practice data comprising one or more of: clinical medical data indexed by the healthcare provider identifiers, prescription data specifying drug prescriptions previously written and indexed by the healthcare provider identifiers and/or medical claims data indexed by the healthcare provider identifiers and storing the data in a shared database;
displaying, in a graphical user interface that is generated by the DSP campaign planning system and provided to one or more client computers, counts of healthcare providers based upon using the first list and matching healthcare provider identifiers in the first list to the practice data;
receiving, from one or more client computers, input specifying one or more filter attributes, and in response, filtering the first list based on the one or more filter attributes to produce a filtered second list representing a target audience of healthcare provider identifiers;
transmitting the filtered second list of healthcare provider identifiers to a DSP service implemented using one or more DSP server computers, the DSP service being programmed to receive a particular healthcare provider identifier, match the particular healthcare provider identifier to the filtered second list, and in response to a match, generate instructions for use by one or more of an advertising exchange, media server and/or media and advertisement display channel to cause presentation of a targeted digital impression on a user computer associated with the particular healthcare provider identifier.

12. The storage media of claim 11, the healthcare provider identifiers comprising any of United States National Provider identifiers (NPIs), CINC values or Practitioner Identifiers.

13. The storage media of claim 11, further comprising instructions which when executed using one or more computers cause the one or more computers to perform receiving, at the one or more computers, for use in association with the practice data, one or more of demographic data, specialty data, best-of-breed address data and/or education data, each being indexed by the healthcare provider identifiers.

14. The storage media of claim 11 wherein the practice data comprises at least one of diagnosis, procedure, or prescription history associated with the healthcare provider identifiers.

15. The storage media of claim 11 wherein the clinical medical data comprises practice and patient statistics related to at least one of HCP specialty, geography, or HCP claim codes.

16. The storage media of claim 11, further comprising instructions which when executed using one or more computers cause the one or more computers to perform repeating, two or more times, the steps of displaying in a graphical user interface that is generated by the DSP campaign planning system and receiving input specifying one or more filter attributes, thereby repeatedly updating the filtered second list according to different filter attributes.

17. The storage media of claim 11 further comprising instructions which when executed using one or more computers cause the one or more computers to perform generating and displaying a graphical user interface that is programmed to receive client computer input specifying filter attributes for clinical data by diagnosis or procedure, for specialty by primary or non-primary specialty, for geography, for deciles of the healthcare provider identifiers, for diagnosis codes, and for procedure codes.

18. The storage media of claim 11, the DSP service being programmed for:
generating instructions for ranking impressions based upon one or more of target HCP practice specialties, target HCP geography, target HCP procedure codes, counts of unique HCPs, counts of unique HCP patients, or estimated numbers of impressions;
generating instructions for submitting bids for purchasing impressions based upon the ranking of the impressions.

19. The storage media of claim 11, the DSP service being programmed for:
generating instructions for ranking impressions based upon one or more of target HCP practice specialties, target HCP geography, target HCP procedure codes, counts of unique HCPs, counts of unique HCP patients, or estimated numbers of impressions;
generating instructions for submitting bids for purchasing impressions based upon the ranking of the impressions;
the method further comprising:
receiving, across a computer network, results of impressions that were generated in response to the digital publicizing instructions;
based upon the results of impressions, revising instructions for purchasing impressions;
transmitting the revised instructions to an advertising exchange server for executing one or more targeted digital impressions.

20. The storage media of claim 11, the particular healthcare provider identifier being received from a cookie, mobile advertising identifier, third-party identity token, or other digital identity data value that is capable of use in digital advertising.
